(19) United States
(12) Patent Application Publication
(10) Pub. No.: US 2014/0004555 A1
(43) Pub. Date: Jan. 2, 2014

(54) Non-Transformed, Immortalized Human T-Lymphocyte Cell-Lines

(71) Applicant: Southern Methodist University, Dallas, TX (US)

(72) Inventors: Robert Harrod, Rowlett, TX (US); Megan M. Romeo, Dallas, TX (US)

(21) Appl. No.: 13/929,328

(22) Filed: Jun. 27, 2013

Related U.S. Application Data
(60) Provisional application No. 61/665,106, filed on Jun. 27, 2012.

(51) Int. Cl. C12N 5/0783

(52) U.S. Cl. CPC C12N 5/0636
USPC 435/29; 435/325; 435/372.3; 435/455

(57) ABSTRACT

The present invention includes a composition and a method of making the same comprising a non-transformed, immortalized T-lymphocyte cell-line, wherein the T lymphocytes are IL-2 dependent and interact with an extracellular matrix.

### CROSS-REFERENCE TO RELATED APPLICATIONS

This application claims benefit of U.S. Provisional Application Ser. No. 61/665,106 filed on Jun. 27, 2012, which is incorporated herein by reference in its entirety.

### STATEMENT OF FEDERALLY FUNDED RESEARCH

This invention was made with U.S. Government support under Contract Nos. 1R15CA139425-01 and 1R15CA158945-01A1, awarded by the National Cancer Institute/National Institutes of Health (NCI/NIH). The government has certain rights in this invention.

### TECHNICAL FIELD OF THE INVENTION

The present invention relates in general to the field of immunology, and more particularly, to a non-transformed, immortalized human T-lymphocyte cell-line for use in biological and pharmacological applications.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jun. 27, 2013, is named SMU1016_SL.txt and is 4,096 bytes in size.

### BACKGROUND OF THE INVENTION

Without limiting the scope of the invention, its background is described in connection with T cell activation.

U.S. Pat. No. 4,704,357, issued to Mitsuya, et al., is directed to Immortalized T-lymphocyte cell line for testing HTLV-III inactivation. Briefly, Mitsuya, et al. teach an immortalized T cell clone, designated ATH8, which was found to be highly sensitive to the cytopathic effect of HTLV-III. The ATH8 T cell clone can be used in mass screening systems to rapidly and easily determine the in vitro capacity of new drugs or other agents to inactivate or inhibit HTLV-III or related cytopathic retroviruses.

### SUMMARY OF THE INVENTION

In one embodiment the present invention includes a composition comprising a non-transformed, immortalized T-lymphocyte cell-line, wherein the T lymphocytes are IL-2 dependent and interact with an extracellular matrix. In one aspect, the T-lymphocyte cell-line supports productive infection and replication by T-tropic HIV. In another aspect, the T-lymphocyte cell-line cells are polyclonal. In another aspect, the T-lymphocyte cell-line cells are monoclonal. In another aspect, the T-lymphocytes are human. In another aspect, T-lymphocytes are transfected with an HTLV-1 p30^{II} oncoprotein. In another aspect, T-lymphocytes are infected with a virus that expressed an HTLV-1 p30^{II} oncoprotein. In another aspect, the T-lymphocytes are infected with a lentivirus that expressed an HTLV-1 p30^{II} oncoprotein. In another aspect, the T-lymphocytes are transfected with an HTLV-1 p30^{II} oncoprotein expressed from the nucleic acid sequences of SEQ ID NO: 1. In another aspect, T-lymphocytes are transfected with an HTLV-1 p30^{II} oncoprotein comprises the acid sequences of SEQ ID NO: 2. In another aspect, the T-lymphocytes are obtained from primary peripheral blood mononuclear cells.

In another embodiment, the present invention also includes a method of making immortalized T cells comprising: obtaining peripheral blood mononuclear cells (PBMCs); transducing the peripheral blood mononuclear cells with a vector engineered to express a translation-optimized version of the HTLV-1 p30^{II} oncoprotein; culturing the transduced PBMCs in the presence of Blasticidin; and passaging the transduced PBMCs beyond crisis in the presence of human recombinant IL-2 until immortalization. In one aspect, the vector is lentiviral. In another aspect, the PBMCs are primary PBMCs. In another aspect, the PBMCs are human. In another aspect, the immortalized T cells are IL-2 dependent and interact with an extracellular matrix. In another aspect, the T-lymphocytes are infected with a virus that expressed an HTLV-1 p30^{II} oncoprotein. In another aspect, the T-lymphocytes are transfected with an HTLV-1 p30^{II} oncoprotein expressed from the nucleic acid sequences of SEQ ID NO: 1. In another aspect, the T-lymphocytes are transfected with an HTLV-1 p30^{II} oncoprotein comprises the acid sequences of SEQ ID NO: 2.

Yet another embodiment of the present invention includes method of evaluating a candidate drug believed to be useful in treating a disease, the method comprising: (a) contacting the candidate drug with a non-transformed, immortalized T-lymphocyte cell-line, wherein the T-lymphocytes are IL-2 dependent, interact with an extracellular matrix; and (b) monitoring the response by the T-lymphocyte cell-line to the candidate drug, wherein a relative and statistically significant activation or suppression of the T-lymphocyte cell-line indicates that the candidate drug is useful in modifying a T-lymphocyte response. In one aspect, the PBMCs are primary PBMCs. In another aspect, the PBMCs are human. In another aspect, the immortalized T cells are IL-2 dependent and interact with an extracellular matrix. In another aspect, the T-lymphocytes are infected with a virus that expressed an HTLV-1 p30^{II} oncoprotein. In another aspect, the T-lymphocytes are transfected with an HTLV-1 p30^{II} oncoprotein expressed from the nucleic acid sequences of SEQ ID NO: 1. In another aspect, the T-lymphocytes are transfected with an HTLV-1 p30^{II} oncoprotein comprises the acid sequences of SEQ ID NO: 2.

### BRIEF DESCRIPTION OF THE DRAWINGS

For a more complete understanding of the features and advantages of the present invention, reference is now made to the detailed description of the invention along with the accompanying figures and in which:

FIGS. 1A-B. Micrographs of the immortalized RomT T-lymphocyte cell-line. FIG. 1A, 10× magnification—the cultures exhibit both semi-adherent and large multicellular aggregate T cell populations (the scale bar represents 100 um). FIG. 1B, 100× magnification—the RomT lymphocytes form multicellular clusters and display oval and elongated morphologies with fibril-like extensions (zoomed image, right panel), which are typical of activated T-lymphocytes (the scale bar represents 20 um).

FIG. 2 has three panels, which include two line drawings that are renderings of original color micrographs that show the HTLV-1 p30^{II} oncoprotein is expressed in the nucleoplasm of immortalized RomT T-lymphocytes. Immunofluorescence-confocal microscopy was performed by staining fixed and permeabilized RomT cells with a mouse monoclonal primary antibody against the Hemagglutinin-tag to detect the p30^{II} (HA-tagged protein). The cells were then stained with a Rhodamine red-conjugated fluorescent anti-mouse secondary antibody (middle panel) hatched vertically, and DAPI (right panel) hatched horizontally. The DIC phase-contrast image is shown for reference (left image).

FIG. 3 includes six panels of rendered line drawings based on original color micrographs that show HTLV-1 p30^{II}(HA)-immortalized RomT lymphocytes (continuously passaged for 32 weeks) that were labeled with bromodeoxyuridine (BrdU) and immunofluorescence microscopy was performed using an Anti-BrdU-FITC conjugated antibody. 7-AAD staining of the DNA is shown for reference.

FIG. 4 shows the nucleotide coding sequence (SEQ ID NO.: 1) and primary amino acid sequence (SEQ ID NO.: 2) of HTLV-1 p30^{II} Opt (HA-tagged).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes the first non-transformed, immortalized human T-lymphocyte cell-line, designated RomT. At present, all commercially-available T cell-lines (e.g., Molt-4, HuT-78, Jurkat, HuT-102, MJG11) are either transformed or were derived from human cancers and do not exhibit normal T-lymphocyte characteristics, and therefore are of limited usefulness for biological research (e.g., immunology, infectious disease, inflammation, oncology) and pharmaceutical drug discovery/screening or pharmacological toxicity analyses. The new RomT T-lymphocyte cell-line exhibits IL-2-dependence and interacts with extracellular matrix (ECM) components. The RomT cell-line can be generated by transducing primary human peripheral blood mononuclear cells (PBMCs) with a lentiviral vector engineered to express a translation-optimized version of the HTLV-1 p30^{II} oncoprotein. In this regard, the RomT T-lymphocyte cell-line is comparable to the 293T human embryonic kidney (HEK) cell-line which expresses the SV40 polyoma virus large T-oncoprotein. The transduced cultures were selected on Blasticidin and repeatedly passaged and expanded beyond crisis in the presence of human recombinant IL-2, until immortalization was established. The RomT lymphocyte cell-line retains many physiological characteristics of normal T cells, and can be used for basic science and biomedical research, including drug candidate screening, as well as for pharmaceutical drug discovery/screening and pharmacological in vitro toxicity testing and can be made from any PBMCs.

There are three general types of cell cultures: (1) Primary—derived from human or animal tissues and organs (pluripotent stem cells and tissue-specific progenitors are included in this category), (2) Immortalized (or continuous)—derived from primary cells which have been engineered to divide and proliferate indefinitely in culture (these cells retain many characteristics of normal primary cells, such as contact-inhibition of growth in the case of adherent fibroblasts), and (3) Transformed—derived from cancerous tissues or oncogenically transformed in vitro by cancer-inducing viruses (these cells do not resemble normal primary cells and behave like tumor cells. Transformed cells exhibit a loss of contact-inhibition, growth factor-independence or reduced requirement for soluble growth factors and serum, and anchorage (ECM)-independent growth (Flint et al, 2004). For most biomedical and pharmaceutical research and development applications (e.g., in vitro efficacy and toxicity testing of pharmacological drug candidates), it is generally desirable to use a cellular background that closely recapitulates normal physiological conditions. While primary cultures most closely resemble the normal tissue microenvironment, there are significant difficulties in obtaining these cells from human or animal tissues and complex regulatory requirements (e.g., Institutional Animal Care & Usage Committees; Human Subjects Research-Institutional Review Boards), and the general difficulties associated with maintaining and growing primary cells in vitro (growth factor- and stromal-dependence), make it difficult to use these cells for most applications. Primary cells have a finite doubling-capacity (usually 40-60 replication cycles) before they undergo crisis and senescence (RA Weinberg, 2007). The use of primary cultures can also introduce significant reproducibility errors, as these cells must be continually re-isolated to conduct multiple experiments.

Therefore, as used herein the term “immortalized” or “continuous” with regard to the cellular characteristics of cell lines derived from primary cells refers to a T-lymphocytes (or T cells) engineered to divide and proliferate indefinitely in culture. These cells retain many characteristics of normal primary cells, such as, e.g., contact-inhibition of growth in the case of adherent cells and IL-2 dependence.

Immortalized cell-lines are generally favored for most biological and pharmaceutical industrial applications, as they can be maintained indefinitely (which reduces the possibility of errors due to reproducibility) and these cells retain many characteristics of normal primary tissues. The use of immortalized cell lines does not require review and approval by IACUC and HSR-IRB committees or federal granting agencies. Immortalized cell-lines are typically generated by treating primary cells with a chemical mutagen, or expressing an oncoprotein of a cancer-inducing virus (e.g., SV40 polyoma virus T-antigen; adenovirus E1A protein. RA Weinberg, 2007; Jat and Sharp, 1986; Velcich and Ziff, 1985). For example, 293T human embryonic kidney (HEK) cells are an adherent cell-line, immortalized by the SV40 polyoma virus large T-oncoprotein and adenovirus sequences, which grows as a single-cell monolayer similar to normal cells (Sena-Esteves et al, 1999). Also, the HFL1 cell-line is an immortalized human fetal lung fibroblast cell-line which exhibits a stable diploid karyotype and grows as a monolayer (Breul et al, 1980); and the 3T3 murine fibroblast cell line was derived from mouse embryonic cells that were repeatedly passaged in vitro (Todaro et al, 1963). The immortalized Hs578Bst breast epithelial cell line was derived from normal mammary epithelium growing adjacent to an invasive ductal carcinoma (Hackett et al, 1977).

Interestingly, although numerous immortalized adherent (e.g., fibroblast; epithelial) cell-lines have been developed, there are currently no non-transformed immortalized T-lymphocyte cell-lines available. The present invention fills a unique niche and represents a novel advancement for biological and pharmacological studies requiring normal T-lymphocyte functions.

Transformed cell-lines are derived from cancerous tissues or cells that were infected with oncogenic transforming viruses (e.g., HTLV-1, murine leukemia virus, mouse mammary tumor virus, Rous sarcoma virus, human papillomavirus). These cells have lost most of their normal characteristics and behave like tumor cells in culture (e.g., exhibit loss of contact-inhibition, and grow as multicellular foci with reduced growth factor-dependence and anchorage-independence). There are several thousand available transformed cell-lines (HeLa cells—an HPV transformed human epithelial carcinoma cell-line is one of the best examples), representative of all types of malignant tissues. Most notably, all of the available T-lymphocyte cell-lines are transformed or were derived from human cancers.

For example, the Jurkat cell-line, which is the most widely used T cell-line in biological and pharmaceutical research applications, was originally derived from an acute lymphoblastic leukemia (Schneider et al, 1977). The HuT-78 and Molt-4 T cell-lines were similarly derived from Sezary syndrome (cutaneous T cell leukemia) and acute lymphoblastic leukemia, respectively (Gootenberg et al, 1981; Minowada et al, 1972). The HuT-102, MJG11, MT-2, and MT-4 T-lymphocyte cell-lines are all HTLV-1-transformed cell-lines that were derived from HTLV-infected acute T cell leukemia/lymphoma (ATLL) patients (Poiesz et al, 1980; Popovic et al, 1983; Kobayashi et al, 1984; Harada et al, 1985).

The present immortalized RomT cell-line invention is a significant improvement over currently available T cell-lines, and provides a near physiological cellular background to support biological (e.g., immunology, viral and bacterial infectious disease, inflammation, and oncology, and screening for drug candidates that can affect the immune response to the same) research and pharmaceutical industrial drug discovery/screening and in vitro toxicity analyses. RomT cells lines can be generated from any PBMCs following the methods disclosed herein.

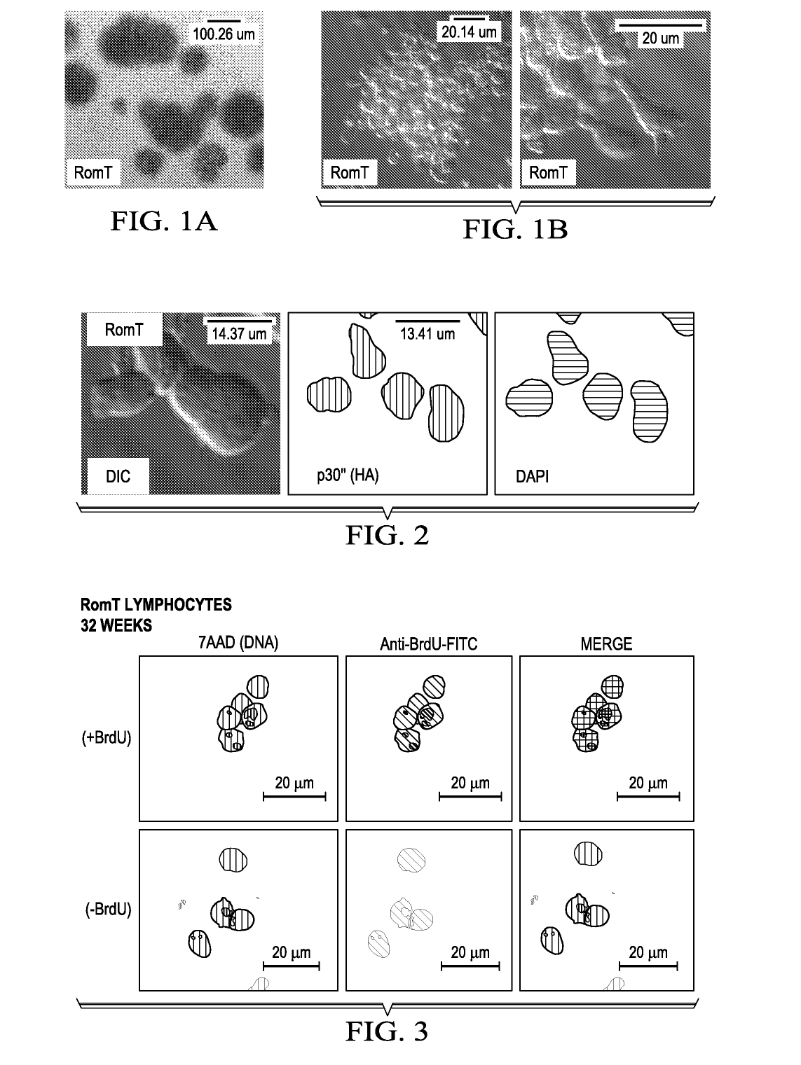

Generation of the immortalized RomT T-lymphocyte cell-line. The RomT T-lymphocyte cell-line was generated by transducing primary human peripheral blood mononuclear cells (PBMCs) isolated from whole-blood with a lentiviral vector, engineered to express a translation-optimized version of the HTLV-1 p30^{II} oncoprotein (HA-tagged. Romeo et al, manuscript in preparation). The present inventors demonstrated previously that the p30^{II} protein activates MYC-dependent transcription (Awasthi et al, 2005). The MYC transcription factor also drives expression of the human telomerase reverse transcriptase (hTERT) gene, which promotes cellular immortalization (Wu et al, 1999; RA Weinberg, 2007). The transduced cultures were selected on Blasticidin in 96-well microtiter plates and repeatedly passaged and expanded beyond crisis in the presence of recombinant human IL-2 (10 U/ml), until immortalization was established. The RomT lymphocytes are IL-2-dependent and also exhibit a requirement for ECM components. Both semi-adherent and suspension cells are observed in culture; and the cells tend to form large multicellular aggregates that closely resemble normal activated T-lymphocytes (FIGS. 1A and 1B). The RomT cells display oval and elongated cellular morphologies with fibril-like extensions, which are typical of activated T cells (FIG. 1B, right panel). The HTLV-1 p30^{II} (HA-tagged) oncoprotein is uniformly expressed in the nucleoplasm of immortalized RomT lymphocytes, as determined by immunofluorescence-confocal microscopy (FIG. 2). Upon withdrawal of hIL-2, the cultures undergo significant apoptosis (programmed cell-death), which further confirms that the cells are immortalized, but not transformed—as IL-2-independence is a generally accepted transformation criterion for T-lymphocytes (Noraz et al, 1998). By contrast, the HTLV-1 transformed T cell-lines, HuT-102, MJG11, MT-2 and MT-4, as well as the acute lymphoblastic leukemia-derived cell-lines, Jurkat, HuT-78 and Molt-4, are all IL-2-independent. The RomT cell-line also exhibits a semi-adherent (anchorage-dependent) growth phenotype and the cells are maintained on ECM-coated cell-ware.

FIGS. 1A-B include micrographs of the immortalized RomT T-lymphocyte cell-line. FIG. 1A, 10× magnification—the cultures exhibit both semi-adherent and large multicellular aggregate T cell populations (the scale bar represents 100 um). FIG. 1B, 100× magnification—the RomT lymphocytes form multicellular clusters and display oval and elongated morphologies with fibril-like extensions (zoomed image, right panel), which are typical of activated T-lymphocytes (the scale bar represents 20 um).

FIG. 2 has three panels, which include two line drawings that are renderings of original color micrographs that show the HTLV-1 p30^{II} oncoprotein is expressed in the nucleoplasm of immortalized RomT T-lymphocytes. Immunofluorescence-confocal microscopy was performed by staining fixed and permeabilized RomT cells with a mouse monoclonal primary antibody against the Hemagglutinin-tag to detect the p30^{II} (HA-tagged protein). The cells were then stained with a Rhodamine red-conjugated fluorescent anti-mouse secondary antibody (middle panel), and DAPI (right panel). The DIC phase-contrast image is shown for reference (left image).

FIG. 3 includes six panels of rendered line drawings based on original color micrographs that show HTLV-1 p30^{II} (HA)-immortalized RomT lymphocytes (continuously passaged for 32 weeks) that were labeled with bromodeoxyuridine (BrdU) and immunofluorescence microscopy was performed using an Anti-BrdU-FITC conjugated antibody. 7-AAD staining of the DNA is shown for reference.

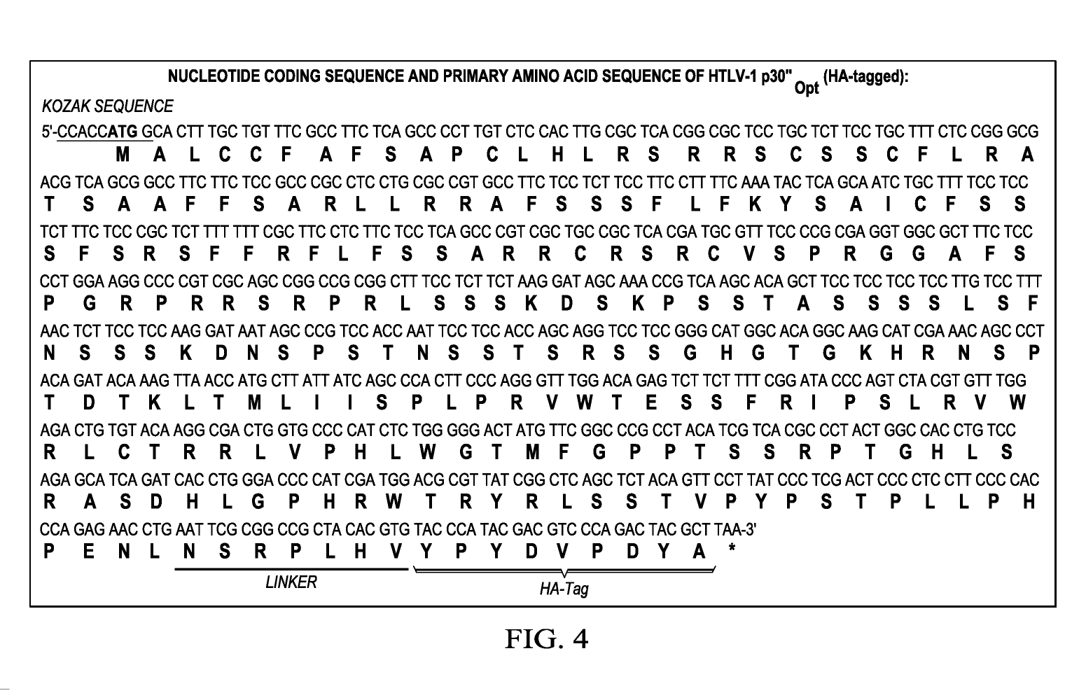

FIG. 4 shows the nucleotide coding sequence (SEQ ID NO.: 1) and primary amino acid sequence (SEQ ID NO.: 2) of HTLV-1 p30^{II} Opt (HA-tagged).

Cell culture. 293FT Fibroblasts (Invitrogen) were grown in Eagle's Minimum Essential Media (MEM, ATCC), 10% fetal bovine serum (FBS; Biowest), 6 mM L-glutamine (Sigma), 4500 mg/ml α-D-glucose (Sigma), 1% penicillin-streptomycin sulfate (Invitrogen-Life Technologies), 500 μg/ml G418 Sulfate (ATCC), and cultured at 37° C. under 5% CO₂. The HT-1080 fibrosarcoma cells (ATCC #: CCL-121) were grown in MEM, 10% FBS, 100 U/ml penicillin, 100 μg/ml streptomycin sulfate, and cultured at 37° C. under 5% CO₂. Molt-4 T-lymphocytes (ATCC #: CRL-1582) were grown in RPMI-1640 (ATCC), 20% FBS, 100 U/ml penicillin, 100 μg/ml streptomycin sulfate, and cultured at 37° C. under 10% CO₂. Buffy-coat, human peripheral blood mononuclear cells (hu-PBMCs) were isolated from whole-blood (obtained with Informed Consent and human subjects research-IRB approval) using Cappel LSM Lymphocyte Separation Medium and centrifugation, and cultured in RPMI-1640, 20% FBS, 100 U/ml penicillin, 100 μg/ml streptomycin sulfate, 2 μg/ml of gentamicin sulfate (Invitrogen), 10 U/ml hIL-2 (Roche) at 37° C. under 10% CO₂.

Lentivirus Production and Engineering the HTLV-1 p30^{II}_{Opt} Gene Insert. The translation-optimized HTLV-1 p30^{II}_{Opt} (HA-tagged) gene insert was engineered and generated by polymerase chain reaction (PCR) amplification of the HTLV-1 p30^{II} (HA-tagged) nucleotide sequence from the pME-CMV-HTLV-1 p30^{II} (HA) expression construct using an HTLV-1 p30^{II} upstream oligonucleotide primer and HA-tag downstream oligonucleotide DNA primer. The translation-optimized HTLV-1 p30^{II}_{Opt} (HA-tagged) nucleotide sequence was engineered by PCR-mutagenesis to contain a consensus Kozak sequence (5′-CCACCATGG-3′) surrounding the ATG_{Met} initiation codon and third position/wobble substitution mutation (CTA_{Leu}CTT_{Leu}), which does not alter the primary amino acid sequence. Polymerase chain reactions were performed using Deep Vent Polymerase (NEB Laboratories) to generate the engineered HTLV-1 p30^{II}_{Opt} (HA) gene insert. The PCR product was sequenced in both directions, cloned into the pCR8/GW/TOPO vector (Invitrogen), and subsequently transformed into competent E. coli OneShot TOP10 bacterial cells (Invitrogen) grown on Lennox agar plates supplemented with 100 μg/ml Spectinomycin. The orientation of the insert was verified by PCR, restriction digest, and sequencing. Using the Invitrogen ViraPower Lentiviral Expression System, pCR8/GW/TOPO HTLV-1 p30^{II}ₒₚₜ (HA) and pLenti6.2/V5-DEST vector were recombined using the LR recombination reaction. The recombination reaction was transformed into OneShot Stb13 competent E. coli bacterial cells (Invitrogen) and grown on Lennox agar plates with 100 μg/ml Ampicillin. The pLenti6.2/V5-DEST HTLV-1 p30^{II}ₒₚₜ (HA) plasmid was verified by restriction digestion. pLenti6.2/V5-DEST HTLV-1 p30^{II}ₒₚₜ (HA) was then transfected into 293FT using Lipofectamine 2000 (Invitrogen) with the ViraPower packaging mix. The supernatant containing extracellular virus particles was harvested after 72 hours. The filtered supernatant was ultracentrifuged at 44,000 rpm at 4° C. for 24 hours on a 20% sucrose-TSE cushion (70% sucrose, 10 mM Tris-Cl (pH 7.5), 100 mM NaCl, 1 mM EDTA). The concentrated virus particles were resuspended in TNE buffer (10 mM Tris-Cl (pH 7.4), 100 mM NaCl, 1 mM EDTA). The virus stock was then titered using HT-1080 fibrosarcoma cells and Molt-4 T-lymphocytes at 10⁰, 10⁻², 10⁻³, 10⁻⁴, 10⁻⁵, 10⁻⁶ dilutions. After 24 hours, 5 μg/ml Blasticidin-HCl was added to the lentiviral-transduced Molt-4 T-lymphocytes; and 10 μg/ml Blasticidin-HCl was added to the transduced HT-1080 fibrosarcoma cells (predetermined by performing a killing curve with Blasticidin-HCl). After 7 days, the transduced cells were immunostained to detect expression of the HA-epitope tag using a mouse monoclonal HA.C5 antibody (Santa Cruz Biotechnology) and immunofluorescence-microscopy was performed.

Immortalization Assay. Primary human PBMCs were plated on BD Falcon 60 mm dishes and transduced with a 10⁻² dilution of the lentiviral stock. After 24 hours, the virus-containing supernatant was removed. Seven days post-transduction, the hu-PBMCs were treated with 5 μg/ml Blasticidin-HCl (Invitrogen). After 24 hours, each 60 mm dish was expanded to a BD Falcon Biocoat poly-D-lysine 96-well plate in media containing 5 μg/ml Blasticidin-HCl. After 14 days, Blasticidin use was discontinued. The transduced hu-PBMCs were expanded to BD Falcon Biocoat Poly-D-Lysine 24-well plates and continuously passaged for more than 9 months.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Romeo M, Ko B, He J, Kim J, Musharof F, Faria A, Kendall K L, Tayeh N, Nelson K A, Lee D, Harrod C K, and Harrod R (2012). Functional interchangeability and transitive homology of the HTLV-1 p30^{II} and HPV 18 E6 oncoproteins. Manuscript in preparation.
Flint S J, Enquist L W, Racaniello V R, and Skalka A M (2004). Virus cultivation, detection, and genetics, In Principles of Virology: Molecular Biology, Pathogenesis, and Control of Animal Viruses, 2^{nd} Edition (ASM Press, Washington, D.C.), pp 26-62.
Weinberg R A (2007). Eternal life: cell immortalization and tumorigenesis, In The Biology of Cancer (Garland Science, New York), pp 357-398.
Jat P S, and Sharp P A (1986). Large T antigens of simian virus 40 and polyomavirus efficiently establish primary fibroblasts. J Virol. 59:746-755.
Velcich A, and Ziff E (1985). Adenovirus E1a proteins repress transcription from the SV40 early promoter. Cell 40:705-716.
Sena-Esteves M, Saeki Y, Camp S M, Chiocca E A, and Breakefield X O (1999). Single-step conversion of cells to retrovirus vector producers with herpes simplex virus-Epstein-Barr virus hybrid amplicons. J Virol. 73:10426-10439.
Breul S D, Bradley K H, Hance A J, Schafer M P, Berg R A, and Crystal R G (1980). Control of collagen production by human diploid lung fibroblasts. J Biol Chem. 255:5250-5260.
Todaro G J, and Green H (1963). Quantitative studies of the growth of mouse embryo cells in culture and their development into established lines. J Cell Biol. 17:299-313.
Hackett A J, Smith H S, Springer E L, Owens R B, Nelson-Rees W A, Riggs J L, and Gardner M B (1977). Two syngeneic cell lines from human breast tissue: the aneuploidy mammary epithelial (Hs578T) and the diploid myoepithelial (Hs578Bst) cell lines. J Natl Cancer Inst. 58:1795-1806.
Schneider U, Schwenk H U, and Bornkamm G (1977). Characterization of EBV-genome negative “null” and “T” cell lines derived from children with acute lymphoblastic leukemia and leukemic transformed non-Hodgkin lymphoma. Int J Cancer 19:621-626.
Minowada J, Onuma T, and Moore G E (1972). Rosette-forming human lymphoid cell lines. I. Establishment and evidence for origin of thymus-derived lymphocytes. J Natl Cancer Inst. 49:891-895.
Gootenberg J E, Ruscetti F W, Mier J W, Gazdar A, and Gallo R C (1981). Human cutaneous T cell lymphoma and leukemia cell lines produce and respond to T cell growth factor. J Exp Med. 154:1403-1418.
Poiesz B J, Ruscetti F W, Gazdar A F, Bunn P A, Minna J D, and Gallo R C (1980). Detection and isolation of type C retrovirus particles from fresh and cultured lymphocytes of a patient with cutaneous T cell lymphoma. Proc Natl Acad Sci USA 77:7415-7419.
Popovic M, Sarin P S, Robert-Gurroff M, Kalyanaraman V S, Mann D, Minowada J, and Gallo R C (1983). Isolation and transmission of human retrovirus (human T cell leukemia virus). Science 219:856-859.
Kobayashi N, Konishi H, Sabe H, Shigesada K, Noma T, Honjo T, and Hatanaka M (1984). Genomic structure of HTLV (human T cell leukemia virus): detection of defective genome and its amplification in MT-2 cells. EMBO J. 3:1339-1343.
Harada S, Koyanagi Y, and Yamamoto N (1985). Infection of HTLV-III/LAV in HTLV-1-carrying cells MT-2 and MT-4 and application in a plaque assay. Science 229:563-566.
Awasthi S, Sharma A, Wong K, Zhang J, Matlock E F, Rogers L, Motloch P, Takemoto S, Taguchi H, Cole M D, Luscher B, Dittrich O, Tagami H, Nakatani Y, McGee M, Girard A M,
Gaughan L, Robson C N, Monnat R J Jr, and Harrod R (2005). A human T cell lymphotropic virus type 1 enhancer of Myc transforming potential stabilizes Myc-TIP60 transcriptional interactions. Mol Cell Biol. 25:6178-6198.
Wu K J, Grandori C, Amacker M, Simon-Vermont N, Polack A, Lingner J, and Dalla-Favera R (1999). Direct activation of TERT transcription by c-MYC. Nat Genet. 21:220-224.
Noraz N, Saha K, Ottones F, Smith S, and Taylor N (1998). Constitutive activation of TCR signaling molecules in IL-2-independent Herpesvirus saimiri-transformed T cells. J Immunol. 160:2042-2045.
Mitsuya H, and Broder S (1985). Immortalized T-lymphocyte cell line for testing HTLV-III inactivation. U.S. patent application No. 06/781,461.

What is claimed is:

1. A composition comprising a non-transformed, immortalized T-lymphocyte cell-line, wherein the T lymphocytes are IL-2 dependent and interact with an extracellular matrix.

2. The composition of claim 1, wherein the T-lymphocyte cell-line supports productive infection and replication by T-tropic HIV.

3. The composition of claim 1, wherein the T-lymphocyte cell-line cells are polyclonal.

4. The composition of claim 1, wherein the T-lymphocyte cell-line cells are monoclonal.

5. The composition of claim 1, wherein the T-lymphocyte cell-line.

6. The composition of claim 1, wherein the T-lymphocytes are human.

7. The composition of claim 1, wherein the T-lymphocytes are transfected with an HTLV-1 p30^{II} oncoprotein.

8. The composition of claim 1, wherein the T-lymphocytes are infected with a virus that expressed an HTLV-1 p30^{II} oncoprotein.

9. The composition of claim 1, wherein the T-lymphocytes are infected with a lentivirus that expressed an HTLV-1 p30^{II} oncoprotein.

10. The composition of claim 1, wherein the T-lymphocytes are transfected with an HTLV-1 p30^{II} oncoprotein expressed from the nucleic acid sequences of SEQ ID NO: 1.

11. The composition of claim 1, wherein the T-lymphocytes are transfected with an HTLV-1 p30^{II} oncoprotein comprises the acid sequences of SEQ ID NO: 2.

12. The composition of claim 1, wherein the T-lymphocytes are obtained from primary peripheral blood mononuclear cells.

13. A method of making immortalized T cells comprising:
obtaining peripheral blood mononuclear cells (PBMCs);
transducing the peripheral blood mononuclear cells with a vector engineered to express a translation-optimized version of the HTLV-1 p30^{II} oncoprotein;
culturing the transduced PBMCs in the presence of Blasticidin; and
passaging the transduced PBMCs beyond crisis in the presence of human recombinant IL-2 until immortalization.

14. The method of claim 13, wherein the vector is lentiviral.

15. The method of claim 13, wherein the PBMCs are primary PBMCs.

16. The method of claim 13, wherein the PBMCs are human.

17. The method of claim 13, wherein the immortalized T cells are IL-2 dependent and interact with an extracellular matrix.

18. The method of claim 13, wherein the T-lymphocytes are infected with a virus that expressed an HTLV-1 p30^{II} oncoprotein.

19. The method of claim 13, wherein the T-lymphocytes are transfected with an HTLV-1 p30^{II} oncoprotein expressed from the nucleic acid sequences of SEQ ID NO: 1.

20. The method of claim 13, wherein the T-lymphocytes are transfected with an HTLV-1 p30^{II} oncoprotein comprises the acid sequences of SEQ ID NO: 2.

21. A method of evaluating a candidate drug believed to be useful in treating a disease, the method comprising:
(a) contacting the candidate drug with a non-transformed, immortalized T-lymphocyte cell-line, wherein the T lymphocytes are IL-2 dependent, interact with an extracellular matrix; and
(b) monitoring the response by the T-lymphocyte cell-line to the candidate drug, wherein a relative and statistically significant activation or suppression of the T-lymphocyte cell-line indicates that the candidate drug is useful in modifying a T-lymphocyte response.

22. The method of claim 21, wherein the PBMCs are primary PBMCs.

23. The method of claim 21, wherein the PBMCs are human.

24. The method of claim 21, wherein the immortalized T cells are IL-2 dependent and interact with an extracellular matrix.

25. The method of claim 21, wherein the T-lymphocytes are infected with a virus that expressed an HTLV-1 p30^{II} oncoprotein.

26. The method of claim 21, wherein the T-lymphocytes are transfected with an HTLV-1 p30^{II} oncoprotein expressed from the nucleic acid sequences of SEQ ID NO: 1.

27. The method of claim 21, wherein the T-lymphocytes are transfected with an HTLV-1 p30^{II} oncoprotein comprises the acid sequences of SEQ ID NO: 2.


SEQUENCE LISTING (2 sequences)

SEQ ID NO: 1 - DNA, 779 bp, Homo sapiens
ccaccatggcactttgctgtttcgccttctcagccccttgtctccacttgcgctcacggcgctcctgctcttcctgctttctccgggcgacgtcagcggccttcttctccgcccgcctcctgcgccgtgccttctcctcttccttccttttcaaatactcagcaatctgcttttcctcctctttctcccgctctttttttcgcttcctcttctcctcagcccgtcgctgccgctcacgatgcgtttccccgcgaggtggcgctttctcccctggaaggccccgtcgcagccggccgcggctttcctcttctaaggatagcaaaccgtcaagcacagcttcctcctcctccttgtcctttaactcttcctccaaggataatagcccgtccaccaattcctccaccagcaggtcctccgggcatggcacaggcaagcatcgaaacagccctacagatacaaagttaaccatgcttattatcagcccacttcccagggtttggacagagtcttcttttcggatacccagtctacgtgtttggagactgtgtacaaggcgactggtgccccatctctgggggactatgttcggcccgcctacatcgtcacgccctactggccacctgtccagagcatcagatcacctgggaccccatcgatggacgcgttatcggctcagctctacagttccttatccctcgactcccctccttccccacccagagaacctgaattcgcggccgctacacgtgtacccatacgacgtcccagactacgcttaa

SEQ ID NO: 2 - protein, 257 aa, Homo sapiens
MALCCFAFSAPCLHLRSRRSCSSCFLRATSAAFFSARLLRRAFSSSFLFKYSAICFSSSFSRSFFRFLFSSARRCRSRCVSPRGGAFSPGRPRRSRPRLSSSKDSKPSSTASSSSLSFNSSSKDNSPSTNSSTSRSSGHGTGKHRNSPTDTKLTMLIISPLPRVWTESSFRIPSLRVWRLCTRRLVPHLWGTMFGPPTSSRPTGHLSRASDHLGPHRWTRYRLSSTVPYPSTPLLPHPENLNSRPLHVYPYDVPDYA